(19) United States
(12) Patent Application Publication
(10) Pub. No.: US 2021/0293678 A1
(43) Pub. Date: Sep. 23, 2021

(54) FOOD TEXTURE EVALUATION DEVICE AND FOOD TEXTURE EVALUATION SYSTEM

(71) Applicant: The Nisshin OilliO Group, Ltd., Tokyo (JP)

(72) Inventors: Tae Satani, Yokohama-shi, Kanagawa (JP); Kazuyoshi Satou, Yokohama-shi, Kanagawa (JP); Yoshihiro Murano, Yokohama-shi, Kanagawa (JP)

(73) Assignee: The Nisshin OilliO Group, Ltd., Tokyo (JP)

(21) Appl. No.: 17/276,494

(22) PCT Filed: Aug. 7, 2019
(86) PCT No.: PCT/JP2019/031060

§ 371 Date: Mar. 16, 2021

(30) Foreign Application Priority Data
Sep. 28, 2018 (JP) 2018-184237

(51) Int. Cl. G01N 3/08; G01N 3/06; G01N 33/02

(52) U.S. Cl. CPC G01N 3/08; G01N 3/06; G01N 33/02; H04N 5/23203; G01N 2203/003; G01N 2203/0087; G01N 2203/0019

(57) ABSTRACT

This food texture evaluation device (2) is provided with: a drive part (23) including a linear motor; a crushing member (22) driven toward a sample (4) by the drive part; a load measuring part (25) that includes a load cell and measures the force applied to a sample stand (21) when the sample is pressed against the crushing member; and a vibration sensor (26) for detecting the vibration generated from the sample to which the force has been applied.

### TECHNICAL FIELD

The present invention relates to a food texture evaluation device and a food texture evaluation system.

### BACKGROUND ART

An evaluation device has been proposed that quantitatively evaluates food texture obtained when food is put in the mouth and chewed (see, e.g., Patent document 1).

The evaluation device described in Patent document 1 includes a rheometer having a vertically movable sample stage, a load cell, and a plunger connected to the load cell, in which contact microphones are provided on the load cell and the plunger.

### CITATION LIST

### Patent Literature

Patent document 1: JP2003-114218A

### SUMMARY OF INVENTION

### Technical Problem

In some evaluation devices, an air cylinder or a hydraulic cylinder is used as a drive source for driving the sample stage or the plunger. In such evaluation devices, air pressure or oil pressure fluctuates with movement of the cylinder and it is thus difficult to control a travel speed of the cylinder or a force (a load) applied to the sample. Particularly, since the travel speed of the cylinder changes also depending on hardness of the sample, it is sometimes difficult to maintain a constant force (load) applied to the sample.

It is an object of the invention to provide a food texture evaluation device and a food texture evaluation system which are capable of controlling a relative velocity between a sample and a crushing member and/or controlling a force applied to the sample.

### Solution to Problem

An embodiment of the invention provides a food texture evaluation device described in [1] to [10] below. In addition, another embodiment of the invention provides a food texture evaluation system described in [11] to [14] below.

[1] A food texture evaluation device, comprising:
a drive unit comprising a linear motor;
a crushing member configured to be driven by the drive unit toward a sample;
a load measuring unit comprising a load cell for measuring a force applied to a sample stage when the sample is pressed by the crushing member; and
a vibration sensor for detecting vibration generated from the sample receiving the force.
   [2] A food texture evaluation device, comprising:
a drive unit comprising a linear motor;
a sample stage configured to place a sample thereon and to be driven by the drive unit toward a crushing member;
a load measuring unit comprising a load cell for measuring a force applied to the sample stage when the sample is pressed by the crushing member; and
a vibration sensor for detecting vibration generated from the sample receiving the force.
   [3] The food texture evaluation device described in [1] or [2] above, wherein the vibration sensor detects sound, which is generated from the sample receiving the force, as the vibration.
   [4] The food texture evaluation device described in [3] above, wherein detection of the sound by the vibration sensor is performed in synchronization with measurement of the force by the load measuring unit.
   [5] The food texture evaluation device described in [1] or [2] above, wherein the crushing member comprises one or more selected from resin, wood, and stainless steel.
   [6] The food texture evaluation device described in [3] above, wherein the crushing member comprises one or more selected from resin, wood, and stainless steel.
   [7] The food texture evaluation device described in [4] above, wherein the crushing member comprises one or more selected from resin, wood, and stainless steel.
   [8] The food texture evaluation device described in [1] or [2] above, further comprising:
a substantially rectangular parallelepiped-shaped gripping member to which the crushing member is removably attached.
   [9] The food texture evaluation device described in [8] above, wherein the crushing member comprises a knob portion protruding in a direction opposite to a direction toward the sample, wherein the gripping member comprises a gripping space extending from one side surface to another side surface opposite thereto and having a shape corresponding to the knob portion, and wherein the knob portion is engaged with the gripping space in such a manner that the crushing member is gripped by the gripping member.
   [10] The food texture evaluation device described in [9] above, further comprising:
a fixing member for fixing the crushing member to the gripping member,
wherein a through-hole is formed on an upper surface of the gripping member so as to penetrate from the upper surface to the gripping space, wherein a groove portion with a predetermined depth is formed on an upper surface of the knob portion, and wherein the fixing member is inserted into the through-hole, is fitted to the groove portion and thereby fixes the crushing member to the gripping member.
   [11] A food texture evaluation system, comprising:
a drive unit comprising a linear motor;
a crushing member configured to be driven by the drive unit toward a sample;
a load measuring unit comprising a load cell for measuring a force applied to a sample stage when the sample is pressed by the crushing member;
a vibration sensor for detecting vibration generated from the sample receiving the force; and
a soundproof box housing the drive unit, the crushing member, the load measuring unit and the vibration sensor.
   [12] The food texture evaluation system described in [11] above, further comprising:
an analytical processing unit configured to analyze data of the load measured by the load measuring unit and data of the vibration detected by the vibration sensor.
   [13] The food texture evaluation system described in [12] above, wherein the analytical processing unit is configured to display data in such a manner that the data of the load and the data of the vibration are aligned to a common time axis or superimposed.
   [14] The food texture evaluation system described in any one of [11] to [13] above, further comprising:
an image capturing unit configured to capture an image of the sample.

### Advantageous Effects of Invention

According to an embodiment of the invention, it is possible to control a relative velocity between a sample and a crushing member, and/or, it is possible to control a force applied to the sample.

### BRIEF DESCRIPTION OF DRAWINGS

FIG. 1 is a schematic diagram illustrating an example configuration of a food texture evaluation system in an embodiment of the present invention.

FIG. 2 is a schematic front view showing an example configuration of a food texture evaluation device in the present embodiment.

FIG. 3A is a front view showing an example of a crushing member.

FIG. 3B is a plan view showing the example of the crushing member.

FIG. 3C is a bottom view showing the example of the crushing member.

FIG. 4 is a left side view showing an example of an actuator.

FIG. 5 is a front view showing an example of a connection portion.

FIG. 6A is a front view showing an example state in which a cover of a soundproof box is close.

FIG. 6B is a front view showing an example state in which the cover of the soundproof box is open.

FIG. 7 is a diagram illustrating an example of sound insulation performance of the soundproof box.

FIG. 8 is a diagram illustrating an example of a result page on which measurement results are displayed.

FIG. 9A is a diagram illustrating an example of change in sound pressure when a cookie is crushed by the food texture evaluation device in the present embodiment.

FIG. 9B is a diagram illustrating an example of change in loudness over time when the cookie is crushed by the food texture evaluation device in the present embodiment.

FIG. 9C is a diagram illustrating an example of change in load over time when the cookie is crushed by the food texture evaluation device in the present embodiment.

FIG. 10A is a diagram illustrating an example of change in sound pressure over time when pastry is crushed by the food texture evaluation device in the present embodiment.

FIG. 10B is a diagram illustrating an example of change in loudness over time when the pastry is crushed by the food texture evaluation device in the present embodiment.

FIG. 10C is a diagram illustrating an example of change in load over time when the pastry is crushed by the food texture evaluation device in the present embodiment.

FIG. 11 is a front view showing a modification of the crushing member.

FIG. 12 is a front view showing another modification of the crushing member.

FIG. 13 is a front view showing still another modification of the crushing member.

FIG. 14A is a front view showing another modification of the crushing member.

FIG. 14B is a plan view showing the other modification of the crushing member.

FIG. 15A is a front view showing another modification of the crushing member.

FIG. 15B is a right side view showing the other modification of the crushing member.

FIG. 15C is a bottom view showing the other modification of the crushing member.

### DESCRIPTION OF EMBODIMENT

An embodiment of the invention will be described below in reference to the drawings. The embodiment below is described as a preferred illustrative example for implementing the invention. Although some part of the embodiment specifically illustrates various technically preferable matters, the technical scope of the invention is not limited to such specific aspects. In addition, a scale ratio of each constituent element shown in each drawing is not necessarily the same as the actual scale ratio.

(Food Texture Evaluation System)

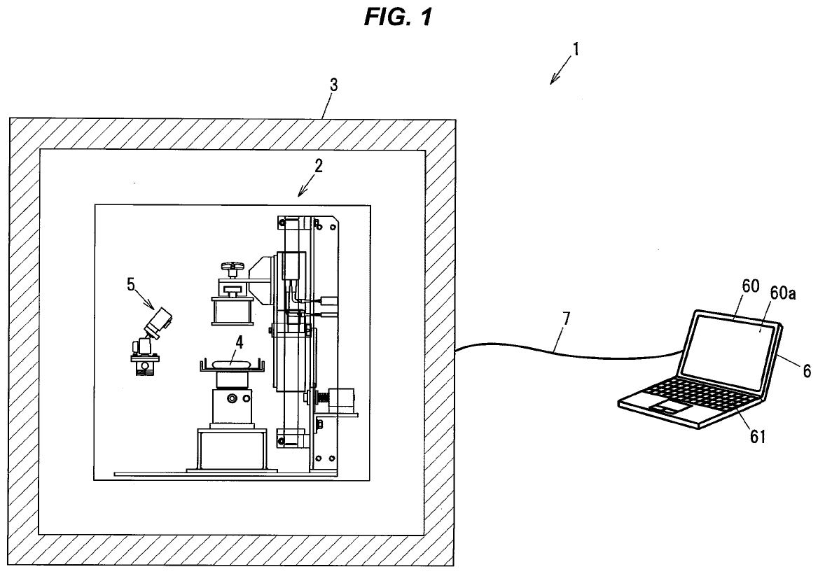

FIG. 1 is a schematic diagram illustrating an example configuration of a food texture evaluation system in an embodiment of the invention. As shown in FIG. 1, a food texture evaluation system 1 has a food texture evaluation device 2 for evaluating food texture of a food sample 4, a soundproof box 3 for blocking ambient sound (hereinafter, also referred to as “background noise” or “noise sound”) entering the food texture evaluation device 2, an image capturing unit 5 which captures an image of the food sample 4, and a control unit 6 connected to the food texture evaluation device 2 and the image capturing unit 5 via a communication portion 7.

The food sample 4 is a sample to be subject to food texture evaluation by the food texture evaluation system 1. The food sample 4 is not specifically limited to a particular food. Examples of the food sample 4 include battered and deep fried foods such as breaded pork cutlet, breaded chicken cutlet, breaded beef cutlet, karaage (lightly floured and deep fried food), deep-fried breaded vegetables and seafood, and tempura, snack confectioneries such as cookie and biscuit, confectionery foods such as rice biscuit, and cereal flour foods made of cereal flour, such as bread, pastry, cake, doughnut, waffle, scone, baumkuchen, cookie, castella (Japanese sponge cake), uiro (traditional Japanese steamed cake made of rice flour and sugar), and manju (traditional Japanese confection with a filling of red bean paste), etc.

The food sample 4 is an example of the sample.

“Food texture” refers to some sensations which are perceived within the mouth when chewing the food sample 4 and can be otherwise expressed as hardness, brittleness, lightness, etc., such as crusty, flaky, crunchy, crispy, crumbly, etc. As another expression, e.g., “crispiness” or “flakiness” may be used to express the food texture.

Next, each element constituting the food texture evaluation system 1 will be described in detail.

### (Food Texture Evaluation Device 2)

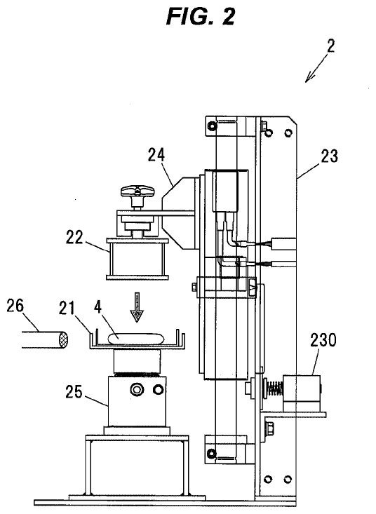

FIG. 2 is a schematic front view showing an example configuration of the food texture evaluation device 2 shown in FIG. 1. As shown in FIG. 2, the food texture evaluation device 2 is provided with a sample stage 21 for placing the food sample 4, a crushing member 22 for crushing the food sample 4 on the sample stage 21, a drive unit 23 for supplying a driving force to vertically drive the crushing member 22 toward the food sample 4 on the sample stage 21, a connection portion 24 connected to the drive unit 23 and transferring the driving force, which is supplied from the drive unit 23, to the crushing member 22, a load measuring unit 25 for measuring a force applied to the sample stage 21 from the food sample 4 when the food sample is pressed by the crushing member 22, and a vibration sensor 26 which detects vibration generated from the food sample 4 during when the food sample 4 is crushed by the crushing member 22.

Although the food texture evaluation device 2 described above is a device in which the sample stage 21 and the load measuring unit 25 are fixed and the crushing member 22 is vertically moved by the drive unit 23, the food texture evaluation device of the invention may be a device in which the crushing member 22 is fixed and the sample stage 21 and the load measuring unit 25 are vertically moved by the drive unit 23.

[Sample stage 21] The sample stage 21 is a platform on which the food sample 4 is placed. The food sample 4 may be placed directly on an inner bottom portion of the sample stage 21 or may be placed on, e.g., a mat (not shown) having a sheet shape with no edge portion and laid on the inner bottom portion of the sample stage 21. The mat has a thickness of, e.g., about 1 mm. To suppress sinking of the food sample 4 and to suppress attenuation of a force transferred from the food sample 4 to the sample stage 21, the mat preferably has a hardness within a predetermined range. Particularly preferably, it does not deform by a force applied to the food sample 4. However, it is not limited to the mat and, e.g., a base board (not shown) may be used.

[Crushing Member 22]

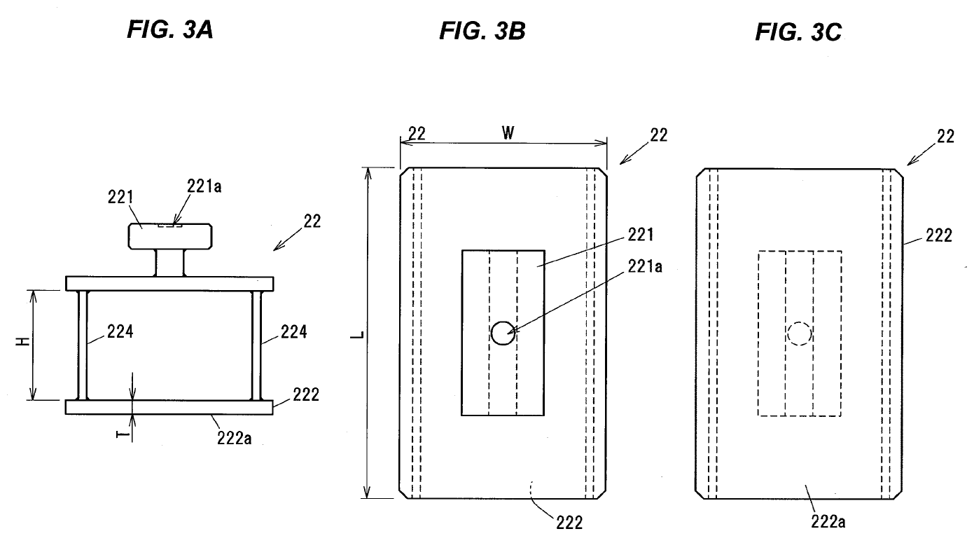

FIG. 3A is a front view showing an example configuration of the crushing member 22, FIG. 3B is a plan view showing the crushing member 22, and FIG. 3C is a bottom view showing the crushing member 22. Note that, since the crushing member 22 has a symmetric property, its back view is identical to the front view and is thus omitted. The crushing member 22 is driven by a driving force generated by the drive unit 23 (see the arrow in FIG. 2) toward the food sample 4 on the sample stage 21. The food sample 4 is pressed in a compression direction when the crushing member 22 after coming into contact with the food sample 4 is further driven.

As shown in each drawing of FIG. 3, the crushing member 22 is provided with a knob portion 221 which protrudes in a direction opposite to a direction toward the food sample 4 (in FIG. 3A, in an upward direction) and has a substantially T-shape when viewed in a vertical cross section, a crushing plate 222 which has a substantially flat bottom surface 222a pressing the food sample 4, and leg members 224 continuous to the knob portion 221 and the crushing plate 222.

The crushing member 22 only needs to have higher strength than food and is formed of one or more selected from resin, wood, and metal material such as stainless steel, etc. In view of suppressing sound (resonant sound, etc.) that the crushing member 22 produces due to contact with the food sample 4 or contact with other constituent elements, the crushing member 22 is preferably formed of resin or wood. On the other hand, in view of corrosive properties or hygroscopic properties, resin or stainless steel is preferable.

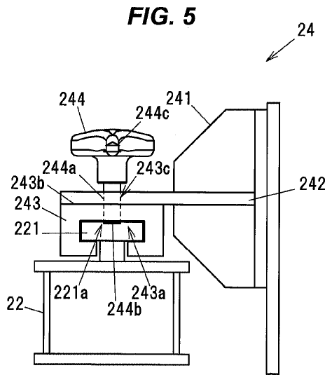

A groove portion 221a into which a tip portion 244b of a fixing member 244 (described later, see FIG. 5) is fitted is formed on an upper surface of the knob portion 221. The shape and size of the groove portion 221a are not specifically limited as long as the tip portion 244b can be fitted thereinto. For example, a depth of the groove portion 221a is not less than 0.5 mm. In addition, the groove portion 221a has a substantially circular shape when viewed from above (see FIG. 3B). A diameter of the groove portion 221a when viewed from above is not limited to a specific range and may be, e.g., 8.5±0.5 mm.

A height H of the leg members 224, a thickness T of the crushing plate 222, a width W of the crushing plate 222 and a length L in a depth direction of the crushing plate 222 may be appropriately adjusted according to the purpose. For example, the height H of the leg members 224 may be 40±5 mm, the thickness T of the crushing plate 222 may be 5±1 mm, the width W of the crushing plate 222 may be 120±10 mm, and the length L of the crushing plate 222 may be 70±5 mm.

[Drive Unit 23]

For example, a ROBO Cylinder or a linear motor may be used as the drive unit 23. Preferably, a linear motor having an actuator 230 is used as the drive unit 23. The linear motor does not use a mechanical drive source to produce thrust and is preferable in that mechanical sound produced by driving can be suppressed. Furthermore, the thrust of the linear motor can be set to any value or a constant value and is preferable in that the force (load) applied to the food sample 4 can be controlled.

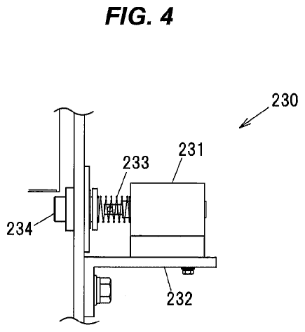

FIG. 4 is a left side view showing an example of the actuator 230. As shown in FIG. 4, the actuator 230 includes a cylindrical solenoid 231, a support plate 232 supporting the solenoid 231, a coil spring 233, and a stopper 234.

An actuator with desired technical specifications may be appropriately selected and used as the actuator 230. As an example, main technical specifications of the actuator 230 used in the present embodiment are summarized in the table below.

TABLE 1
Main technical specifications of Actuator 230
Main technical specifications of Actuator 230

| | | |
|---|---|---|
| 1 | Rated thrust | 118N (532N at maximum) |
| 2 | Stroke length | 65 ± 5 mm |
| 3 | Driving speed | 2.5 mm/s-150 mm/s |

The driving speed here is a relative velocity between the crushing member and the sample (food sample). For example, it means a speed of the driven crushing member 22. The driving speed includes both a speed achieved when the crushing member 22 is not in contact with the food sample 4 and a speed achieved when the crushing member 22 is in contact with the food sample 4. Hereinafter, the driving speed when the crushing member 22 is pressing the food sample 4 is particularly also referred to as “a pressing speed”.

[Connection Portion 24]

FIG. 5 is a front view showing an example of the connection portion 24. As shown in FIG. 5, the connection portion 24 has a bracket 241 connected to the drive unit 23, a coupling plate 242 coupled to the bracket 241, a gripping member 243 for gripping the crushing member 22, and the fixing member 244 for fixing the position of the crushing member 22 which is gripped by the gripping member 243.

The gripping member 243 is a member having a substantially rectangular parallelepiped-shaped. A gripping space 243a is formed in the gripping member 243 so as to extend from one side surface to another side surface opposite thereto. The gripping space 243a is an example of the space.

The gripping space 243a extends from the one side surface to the other side surface in a longitudinal direction in the case of the gripping member 243 shown in FIG. 5 but may extend from one side surface to another side surface in a lateral direction. In addition, the gripping space 243a may or may not penetrate inside the gripping member 243.

In other words, the gripping member 243 is a member which includes a pair of arms which face each other and have a substantially L-shape in a side view shown in FIG. 5. The gripping member 243 may be fixed to, e.g., a bottom side of the coupling plate 242 using a screw(s) (not shown), etc.

The gripping space 243a has a shape corresponding to the knob portion 221 of the crushing member 22. In particular, as shown in FIG. 5, a vertical cross section of the gripping space 243a, i.e., a shape of an opening on the one side surface is substantially the same shape as a vertical cross section of the knob portion 221 of the crushing member 22. Then, a length in a depth direction of the gripping member 243 (a depth length in a direction perpendicular to the plane of the paper of FIG. 5) corresponds to the length of the knob portion 221 of the crushing member 22. Such a gripping space 243a is formed to allow the gripping member 243 to engage the knob portion 221 of the crushing member 22.

In addition, a screw hole 243c is formed at substantially the center of an upper surface 243b of the gripping member 243 so as to penetrate from the upper surface 243b to the gripping space 243a provided inside the gripping member 243. A main body 244a of the fixing member 244 is inserted into the screw hole 243c. The screw hole 243c is an example of the through-hole. When the gripping member 243 is fixed to the coupling plate 242 as shown in FIG. 5, the screw hole 243c also passes through the coupling plate 242.

The fixing member 244 only needs to have a structure that the main body 244a of the fixing member (the tip portion 244b of the fixing member) can move vertically. The fixing member 244 is provided with, e.g., the main body 244a having a substantially cylindrical shape, the tip portion 244b fitted to the groove portion 221a formed on an upper surface of the knob portion 221 of the crushing member 22, and a handle portion 244c for rotating the fixing member 244 about the center axis of the main body 244a.

A screw thread (not shown) is formed on a side surface of the main body 244a, and by turning the handle portion 244c, the main body 244a is inserted into the screw hole 243c of the gripping member 243 or withdrawn from the screw hole 243c by an amount according to a twist of the handle portion 244c.

The knob portion 221 is removably attached to the gripping member 243 of the connection portion 24. In particular, attachment may be done by inserting the knob portion 221 of the crushing member 22 into the gripping space 243a through an opening formed on one side surface constituting the gripping member 243. In this manner, the knob portion 221 of the crushing member 22 engages the gripping space 243a and the crushing member 22 is thereby gripped by the gripping member 243.

In this regard, in case that the gripping space 243a completely penetrates through, attachment can be done by inserting the knob portion 221 of the crushing member 22 in any of near, far, right, left directions when viewed from the front (a direction from the near side to the far side or vice versa in the drawing of FIG. 5, or a direction from left side to the right side or vice versa in the drawing of FIG. 5).

The knob portion 221 of the crushing member 22 is positioned with respect to the gripping member 243 by aligning the substantially circular groove portion 221a, which is formed on the upper surface of the knob portion 221 of the crushing member 22, with the screw hole 243c, which is formed on the upper surface of the gripping member 243, in a state in which the knob portion 221 is attached to the gripping member 243.

Then, when the fixing member 244 is further inserted downward by operating the handle portion 244c in a state in which the groove portion 221a is aligned with the screw hole 243c, the tip portion 244b fits in the groove portion 221a and the crushing member 22 is thereby fixed to the gripping member 243. The bottom surface of the groove portion 221a may be pressed by the tip portion 244b to provide firmer fixation.

[Load measuring unit 25] The load measuring unit 25 measures a force, which is applied to the sample stage 21 during when the food sample 4 is pressed by the crushing member 22 in a downward direction in the drawing (see FIG. 2), as a load (kg) applied to the load measuring unit 25.

For example, a known sensor such as an electromagnetic sensor or a load cell sensor may be used as the load measuring unit 25. Preferably, a load cell-type load sensor is used as the load measuring unit 25. The load cell is suitable for size reduction, hence, preferable. In the present embodiment, e.g., a load cell (FIT7A) from HBM Company is used as the load measuring unit 25.

[Vibration Sensor 26]

The vibration sensor 26 detects vibration generated when the food sample 4 is crushed by the crushing member 22. The vibration sensor 26 is preferably arranged so as not to overlap an edge of the sample stage 21, and is preferably positioned so as to face the food sample 4 from, e.g., diagonally above the edge of the sample stage 21. The vibration sensor 26 detects vibration of air or vibration of any one or more of the crushing member 22, the sample stage 21, etc. To detect, e.g., vibration of air, a sound detection sensor (i.e., a “microphone”) can be used as the vibration sensor 26. Meanwhile, to detect vibration of the crushing member 22, the sample stage 21, etc., an accelerometer or a super high-speed camera may be used. Next, a case where a microphone is used as the vibration sensor 26 will be described as an example.

[Soundproof Box 3]

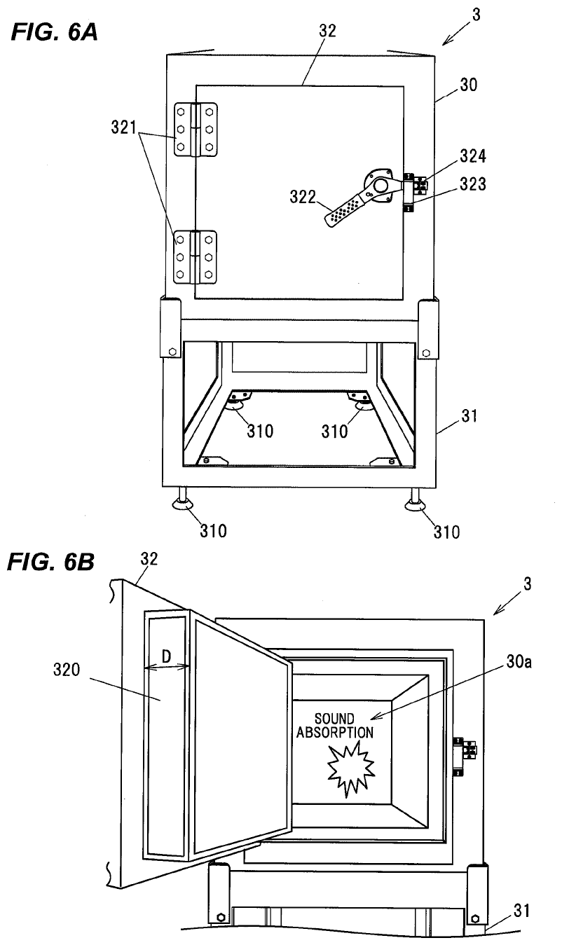

FIG. 6A is a front view showing a state in which a cover of the soundproof box 3 is close, and FIG. 6B is a front view showing a state in which the cover of the soundproof box 3 is open. The soundproof box 3 has a function of blocking external ambient sound and also has a function of absorbing noise sound such as mechanical sound produced by the food texture evaluation device 2. That is, the soundproof box 3 in the present embodiment is an anechoic box. Next, the soundproof box 3 as an anechoic box will be described.

As shown in FIG. 6A, the soundproof box 3 is provided with a housing portion 30 for housing the food texture evaluation device 2, a leg portion 31 supporting the housing portion 30, and a cover (lid) 32 sealing the housing portion 30.

As shown in FIG. 6B, a substantially rectangular parallelepiped-shaped housing space 30a for housing the food texture evaluation device 2 is formed in the housing portion 30. The leg portion 31 is provided with, e.g., plural (e.g., four) casters (wheels)/stoppers 310. An anti-vibration rubber (not shown) is further provided at a contact portion between the housing portion 30 and the leg portion 31.

The cover 32 is provided with hinge members 321 which fix the cover 32 to one side of a surface of the housing portion 30 in an openable and closable manner, a substantially J-shaped grip 322, a door stopper 323 attached to the surface of the housing portion 30, and a door sensor 324 which detects that the cover 32 is closed.

A sound-absorbing material 320 is provided in such a manner that inner surfaces of the housing space 30a are covered with the sound-absorbing material 320 when the cover 32 is closed. The cover 32 has the sound-absorbing material 320 on an inner surface on the housing space 30a side. The sound-absorbing material 320 is formed of, e.g., a cotton-like material made of glass fibers (e.g., glass wool material). A thickness D of the sound-absorbing material 320 is not specifically limited and may be 10±1 cm.

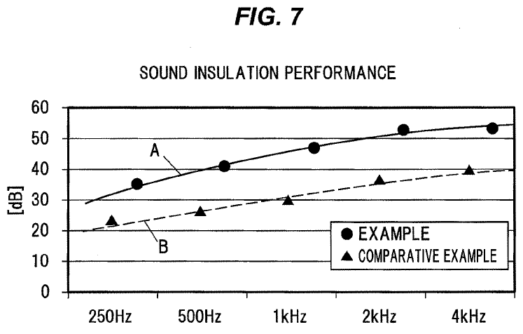

FIG. 7 is a diagram illustrating an example of sound insulation performance of the soundproof box 3. In FIG. 7, the horizontal axis indicates frequency (Hz) of sound and the vertical axis indicates the sound intensity level difference (dB). The result A indicated by a solid line shows an example of sound insulation performance of the soundproof box 3 in the present embodiment, and the result B indicated by a dashed line shows an example of sound insulation performance of a soundproof box in Comparative Example. In Comparative Example, a soundproof box composed of a transparent glass window and a wooden housing portion (not shown) was used.

As shown in FIG. 7, the sound insulation performance of the soundproof box 3 in the present embodiment is higher than the soundproof box in Comparative Example at any frequency. The comparative results of the sound intensities at each frequency are summarized in Table 2 below.

TABLE 2
Comparative results of Sound intensities

| | Sound intensity level difference (dB) | | |
|---|---|---|---|
| | (Sound insulation performance) | | |
| Frequency | Comparative | | Improvement rate |
| (Hz) | Example | Example_{[s1]} | (%) |
| 250 Hz | 23 | 35 | 52 |
| 500 Hz | 26 | 41 | 58 |
| 1 kHz | 29 | 47 | 62 |
| 2 kHz | 36 | 53 | 47 |
| 4 kHz | 39 | 53 | 36 |

In addition, background noise inside the housing portion 30 was suppressed from about 36 dB of the soundproof box in Comparative Example to about 22 dB of the soundproof box in the present embodiment (about 37% of suppression rate) even though it is not shown in the drawing.

(Image Capturing Unit 5)

The image capturing unit 5 captures an image of the food sample 4. The image capturing unit 5 is fixed inside the housing space 30a of the soundproof box 3 by a support member (not shown). A known image sensor may be used as the image capturing unit 5. The image capturing unit 5 may take a still image or may take a moving image. In addition, the image capturing unit 5 may capture an image of the food sample 4 before being crushed by the crushing member 22, or may capture an image of the food sample 4 during being crushed by the crushing member 22, or may capture an image of the food sample 4 after being crushed by the crushing member 22. The captured data is transmitted to the control unit 6 through the communication portion 7.

(Control Unit 6)

For example, a personal computer or a mobile information terminal such as tablet terminal or multifunctional mobile phone (smartphone) may be used as the control unit 6. As shown in FIG. 1, the control unit 6 has a display portion 60 including a display screen 60a for displaying information, and an input portion 61 realized by a keyboard or a mouse, etc., used to input information.

The control unit 6 controls the drive unit 23 of the food texture evaluation device 2 to drive the crushing member 22 to crush the food sample 4. The control unit 6 also controls operations of the load measuring unit 25 and the vibration sensor 26 to perform load measurement and sound detection, and acquires data thereof. The control unit 6 also controls the image capturing unit 5 to capture an image of the food sample 4. The control unit 6 also controls a temperature of the entire food texture evaluation system 1.

The control unit 6 also controls so that measurement of the load applied to the food sample 4 is performed in synchronization with detection of sound generated by crushing. The term “in synchronization with” means to perform substantially simultaneously. The term “substantially simultaneously” is not limited to performing at exactly the same time, but also includes the case where there is a time lag of around a predetermined time (e.g., several milliseconds to several seconds).

The acquired data of load and vibration are analyzed by an analytical processing unit. The analytical processing unit can be installed either inside the food texture evaluation system 1 or outside the food texture evaluation system 1, and the control unit 6 also serves as the analytical processing unit in the configuration in which the analytical processing unit is installed inside the food texture evaluation system 1.

The analytical processing unit analyzes the data of load and the data of vibration and calculates physical quantities such as sound pressure level indicating sound intensity, or analytical values such as loudness, loudness level, sharpness, roughness (hereinafter, also referred to as “psychoacoustic evaluation quantities”). The analytical processing unit may also combine a physical quantity/quantities with a psychoacoustic evaluation quantity/quantities and calculate an index indicating a correlation with food texture.

“Loudness” (unit: sone) is loudness of sound perceived by human, i.e., “perception of sound volume” and the stationary sound is standardized by ISO 532B. One sone is the loudness of sound at a predetermined frequency (e.g., 1,000 Hz) with a predetermined sound pressure level (e.g., 40 dB).

“Loudness level” (unit: phon) is a logarithmic representation of loudness and is the same as the above-described loudness in terms of the fact that it expresses loudness of sound perceived by human, i.e., “perception of sound volume”. The loudness level (L) can be expressed by the following relation (1):

L=10×log 2(N)+40 (1)

where N is the loudness.

“Sharpness” (unit: acum) is so-called “perception of painful (high-pitched) sound”, which is perceived when the balance between low-frequency and high-frequency sounds is biased toward the high-frequency sound. It is an evaluation quantity depending on a frequency component and indicates the spectral balance between low frequency and high frequency.

“Roughness” (unit: asper) is so-called “perception of annoying sound (static, vibrating noise)”, which is perceived when short periodic fluctuations of loudness occur. It is an evaluation quantity of roughness perception occurred when not capable of perceiving quick fluctuations of sound.

The above-described psychoacoustic evaluation quantities such as loudness, loudness level, sharpness, roughness can be calculated by using, e.g., a sound quality evaluation software WS-5160 (manufactured by Ono Sokki Co., Ltd.). This software is installed on, e.g., the control unit 6.

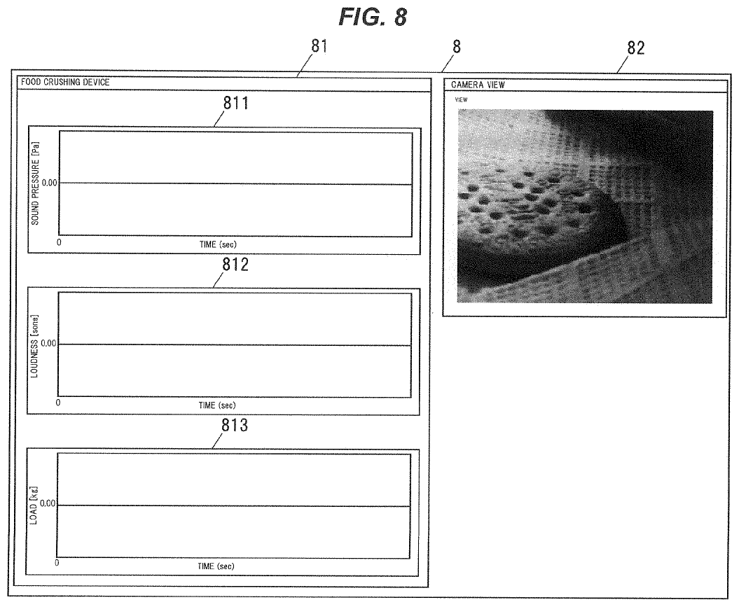

FIG. 8 is a diagram illustrating an example of a result page on which measurement results are displayed. A result page 8 is controlled by the control unit 6 so as to be displayed on the display screen 60a. As shown in FIG. 8, the result page 8 includes a data display field 81 in which various data obtained by the load measuring unit 25 and the vibration sensor 26 (microphone) are displayed on the aligned time-series graphs, and an image display field 82 in which image information such as a photograph (still image) or a moving image, etc., of the food sample 4 captured by the image capturing unit 5 is displayed. The image information may be updated in real time.

For example, a sound pressure graph 811 showing time series data of the sound pressure obtained from analysis of data of sound picked by the microphone, a flakiness graph 812 showing time series data of the psychoacoustic evaluation quantity calculated by the control unit 6, and a load graph 813 showing time series data of the load obtained by the load measuring unit 25, etc., are aligned and displayed on the data display field 81. The graphs 811 to 813 may be displayed on top of each other. In FIG. 8, a graph showing time series data of loudness is shown as an example of the flakiness graph 812.

A measurer can perform an intended control by operating the control unit 6 while referring to the image information displayed in the image display field 82 even when the inside of the soundproof box 3 cannot be visually checked such as when, e.g., the cover 32 of the soundproof box 3 is closed.

(Communication Portion 7)

The communication portion 7 is for transmission and reception of electrical signals which may be wireless or by wire. Alternatively, a network may be used as the communication portion 7, and it is possible to use, e.g., local area network (LAN), wide area network (WAN), internet, intranet, etc.

### EXAMPLES

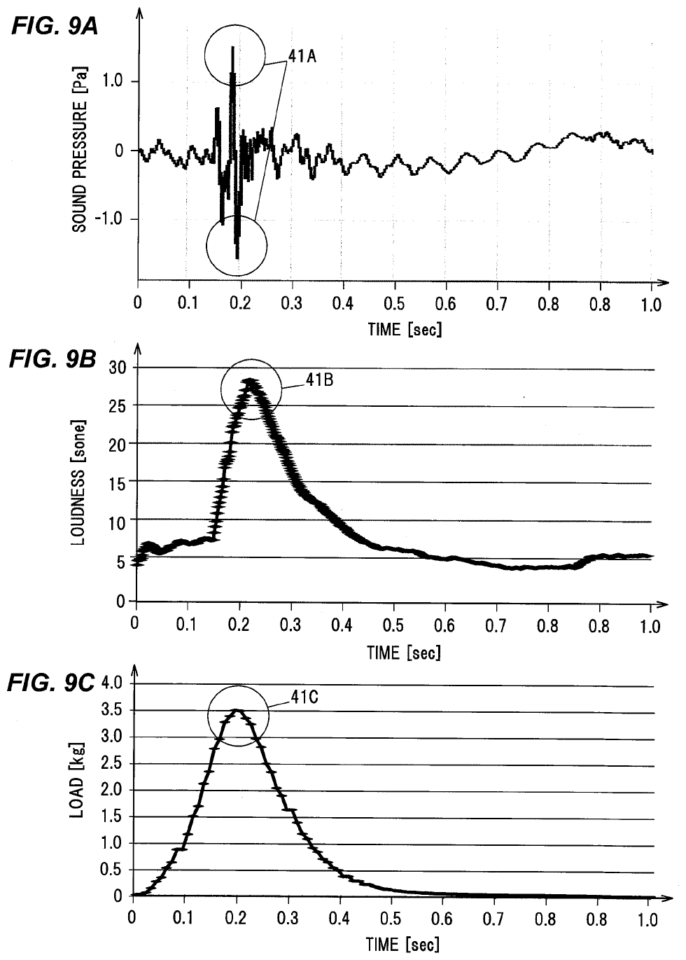

Next, an example of the result of measurement on the food sample 4 by the food texture evaluation device 2 will be described in reference to FIGS. 9A-C and 10A-C.

### [Cookie]

FIG. 9A shows a change in sound pressure over time when a cookie as the food sample 4 is crushed, FIG. 9B shows a change in loudness over time, and FIG. 9C shows a change in load over time. The horizontal axis in each graph indicates common time. The loudness here is an example of sound information. Loudness level, sharpness, roughness described above may be shown instead of loudness, or loudness level, sharpness, roughness, etc., may be further displayed in addition to loudness.

The measurement results shown in each drawing of FIG. 9 are results obtained when a cookie was crushed using the crushing member 22 having a single tooth with an acute-angled edge (described later, see FIG. 12) at a thrust (correlated with the force applied to the sample) by the drive unit 23 of about 10 kg, a driving speed (i.e., a pressing speed) of 30 mm/sec and a pressing distance of 65 mm. The cookie was placed on a mat with a thickness of about 1 mm (not shown) laid on the sample stage 21. The recording range was 316 mV.

As shown in FIG. 9A, as a result of crushing under the above conditions, sound pressure peaks 41A occurred around about 0.2 seconds. Then, as shown in FIG. 9B, a loudness peak 41B corresponding to the sound pressure peaks 41A occurred around about 0.2 seconds. Likewise, as shown in FIG. 9C, a load peak 41C corresponding to the sound pressure peaks 41A occurred around about 0.2 seconds. Based on combination of the results shown in FIGS. 9A-C, it is considered that the cookie was instantaneously broken by the crushing member 22 within time corresponding to the width of the loudness peak 41B or the load peak 41C (within about 0.2 seconds to 0.3 seconds).

[Pastry]

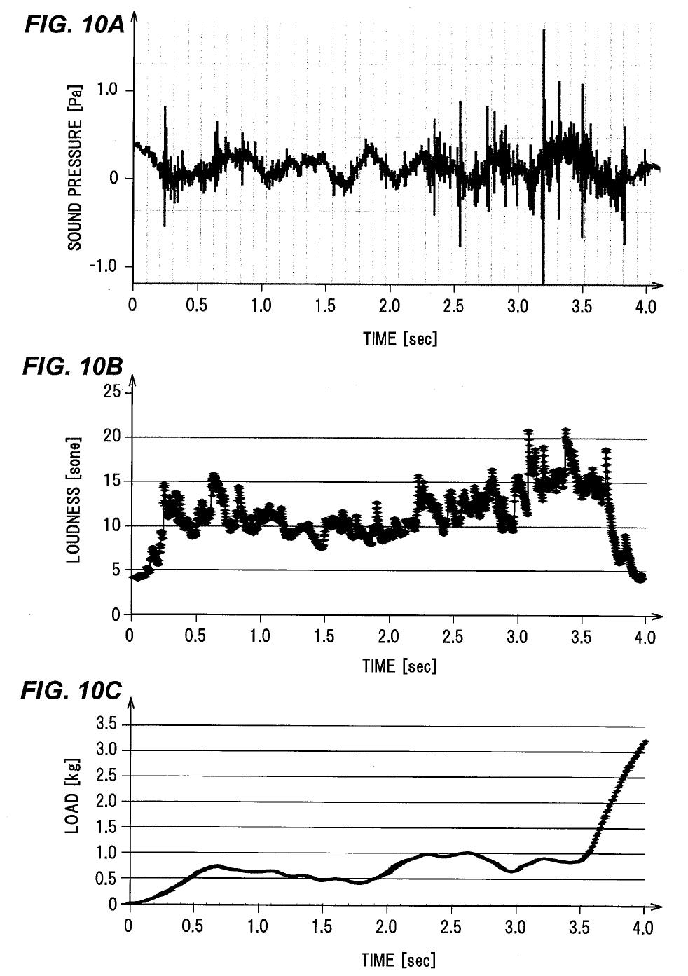

FIG. 10A shows a change in sound pressure over time when a pastry as the food sample 4 is crushed, FIG. 10B shows a change in loudness over time, and FIG. 10C shows a change in load over time. The horizontal axis in each graph indicates common time.

The measurement results shown in each drawing of FIG. 10 are results obtained when a pastry placed on a mat with a thickness of about 1 mm laid on the sample stage 21 was crushed using the crushing member 22 having a single tooth with an acute-angled edge (see FIG. 12) at a thrust (correlated with the force applied to the sample) by the drive unit 23 of about 10 kg, a driving speed (i.e., a pressing speed) of 10 mm/sec and a pressing distance of 65 mm.

The recording range was 100 mV. A baked pastry having a rectangular parallelepiped shape with a size of an average width of about 71 mm, an average length of about 55 mm and an average height of about 37 mm was used.

As shown in FIGS. 10A-C, information of sound pressure and loudness of sound generated by the pastry and the load applied to the pastry at the time of crushing can be substantially simultaneously obtained. Thus, the measurer can comprehensively evaluate the characteristics, such as food texture, of the food sample 4 by combining information of the sound and information of the load which are obtained substantially simultaneously.

### Function and Effects of the Embodiment

By using a linear motor as a drive source and making the crushing member 22 to generate a driving force as described above, it is possible to control the force applied to the food sample 4 regardless of properties, such as hardness, etc., of the food sample 4. Therefore, it is possible to improve reproducibility of the force applied to the food sample 4 as compared to a conventional configuration such as, e.g., air cylinder which uses air pressure as a drive source.

In addition, by using a linear motor as a drive source, it is easier to control the pressing speed of the crushing member 22 as compared to a conventional technique. Therefore, it is possible to, e.g., crush a thin food sample 4 at a low speed or to gradually apply a constant large force to a hard food sample 4.

In addition, by substantially simultaneously measuring the force applied to the sample stage 21 and sound produced by the thin food sample 4, it is possible to obtain data showing a relation between the force applied to the sample stage 21 via the food sample 4 and the sound produced by the food sample 4. By comparing the load data to the sound data in real time, the crush/deformation point of the food sample 4 is clear from the sound data and it is possible to check a change over time of data of the load during crushing/deformation.

<Modifications>

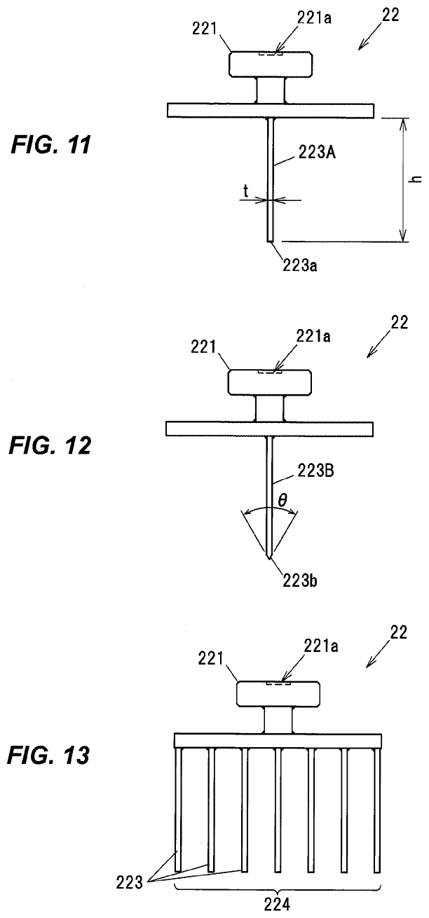

Next, modifications of the crushing member 22 will be described in reference to FIGS. 11 to 15A-C. Each of the crushing members 22 shown in FIGS. 11 to 14A, B has the symmetric property and some portions are omitted in the drawings.

[Modification 1-1]

FIG. 11 is a front view showing a modification of the crushing member 22. As shown in FIG. 11, the crushing member 22 may have a single piece of tooth (hereinafter, also simply referred to as “a single tooth”) instead of the crushing plate 222.

A tip portion 223a of a single tooth 223A has a substantially flat shape. A thickness t and a height h of the single tooth 223A may be appropriately changed according to the purpose.

Preferably, the single tooth 223A has substantially the same thickness as an average thickness of a human tooth (e.g., a front tooth). The thickness t of the single tooth 223A may be, e.g., 2±0.2 mm. The height h of the single tooth 223A may be, e.g., 45±5 mm.

[Modification 1-2]

FIG. 12 is a front view showing another modification of the crushing member 22. As shown in FIG. 12, a tip portion 223b of a single tooth 223B may protrude in an acute angle shape. An angle θ of the tip portion 223b in the vertical cross section shown in FIG. 12 may be appropriately adjusted and may be, e.g., 60±5°.

[Modification 1-3]

FIG. 13 is a front view showing another modification of the crushing member 22. The number of the single teeth 223A is not limited to one, and plural (e.g., seven) teeth 223A may be provided as shown in FIG. 13. The number of the single teeth 223A may be appropriately set according to the purpose. In addition, the configuration is not limited to the example shown in FIG. 13. The configuration may be such that plural teeth 223B each having the tip portion 223b protruding in an acute angle shape shown in FIG. 12 may be provided or such that the single tooth(teeth) 223A having the substantially flat tip portion 223a may be mixed.

[Modification 2]

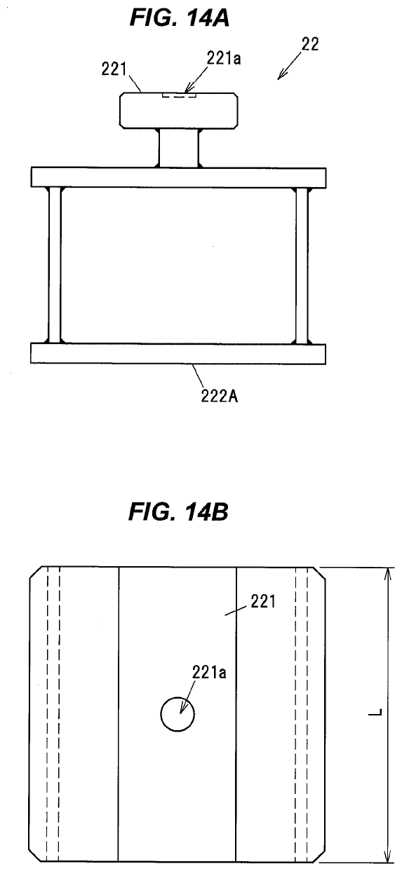

FIG. 14A is a front view showing another modification of the crushing member 22 and FIG. 14B is a plan view showing the modification of the crushing member 22. Since a front view of the crushing member 22 shown in FIG. 14A is the same as that shown in FIG. 3A, the detailed description therefor will be omitted. A length L in a depth direction of a crushing plate 222A (see FIG. 14B) may be substantially the same as the length of the knob portion 221.

[Modification 3]

FIG. 15A is a front view showing another modification of the crushing member 22, FIG. 15B is a right side view showing the modification of the crushing member 22, and FIG. 15C is a bottom view showing the modification of the crushing member 22. As shown in each drawing of FIG. 15, the configuration may be such that the single tooth 223B is removably attached to the crushing member 22.

In particular, it may be configured such that a support 225 with screw holes 225a formed at predetermined positions is provided and the single tooth 223B is fixed to the support 225 using a predetermined number (e.g., three) of screws 226.

Although the embodiments of the invention have been described, the invention is not intended to be limited to the embodiments described above, and the various kinds of modifications can be implemented without departing from the gist of the invention. For example, the food texture evaluation device 2 when placed in a soundproof room or an anechoic chamber does not necessarily need to be housed in the soundproof box 3. In addition, the configuration may be such that, e.g., the food texture evaluation device 2 is provided with the image capturing unit 5. Furthermore, for example, calculation of sound pressure level or psychoacoustic evaluation quantities is not necessarily be performed by the analytical processing unit (the control unit 6) and may be performed by the vibration sensor 26.

### INDUSTRIAL APPLICABILITY

Provided are a food texture evaluation device and a food texture evaluation system which are capable of controlling a relative velocity between a sample and a crushing member and/or controlling a force applied to the sample.

### REFERENCE SIGNS LIST

1: food texture evaluation system
2: food texture evaluation device
21: sample stage
22: crushing member
221: knob portion
221a: groove portion
23: drive unit
243: gripping member
243a: gripping space
243c: screw hole
244: fixing member
25: load measuring unit
26: vibration sensor
3: soundproof box
5: image capturing unit
6: control unit

1. A food texture evaluation device, comprising:
a drive unit comprising a linear motor;
a crushing member configured to be driven by the drive unit toward a sample;
a load measuring unit comprising a load cell for measuring a force applied to a sample stage when the sample is pressed by the crushing member; and
a vibration sensor for detecting vibration generated from the sample receiving the force.

2. A food texture evaluation device, comprising:
a drive unit comprising a linear motor;
a sample stage configured to place a sample thereon and to be driven by the drive unit toward a crushing member;
a load measuring unit comprising a load cell for measuring a force applied to the sample stage when the sample is pressed by the crushing member; and
a vibration sensor for detecting vibration generated from the sample receiving the force.

3. The food texture evaluation device according to claim 1, wherein the vibration sensor detects sound, which is generated from the sample receiving the force, as the vibration.

4. The food texture evaluation device according to claim 3, wherein detection of the sound by the vibration sensor is performed in synchronization with measurement of the force by the load measuring unit.

5. The food texture evaluation device according to claim 1, wherein the crushing member comprises one or more selected from resin, wood, and stainless steel.

6. The food texture evaluation device according to claim 3, wherein the crushing member comprises one or more selected from resin, wood, and stainless steel.

7. The food texture evaluation device according to claim 4, wherein the crushing member comprises one or more selected from resin, wood, and stainless steel.

8. The food texture evaluation device according to claim 1, further comprising:
a substantially rectangular parallelepiped-shaped gripping member to which the crushing member is removably attached.

9. The food texture evaluation device according to claim 8, wherein the crushing member comprises a knob portion protruding in a direction opposite to a direction toward the sample,
wherein the gripping member comprises a gripping space extending from one side surface to another side surface opposite thereto and having a shape corresponding to the knob portion, and
wherein the knob portion is engaged with the gripping space in such a manner that the crushing member is gripped by the gripping member.

10. The food texture evaluation device according to claim 9, further comprising:
a fixing member for fixing the crushing member to the gripping member,
wherein a through-hole is formed on an upper surface of the gripping member so as to penetrate from the upper surface to the gripping space,
wherein a groove portion with a predetermined depth is formed on an upper surface of the knob portion, and
wherein the fixing member is inserted into the through-hole, is fitted to the groove portion and thereby fixes the crushing member to the gripping member.

11. A food texture evaluation system, comprising:
a drive unit comprising a linear motor;
a crushing member configured to be driven by the drive unit toward a sample;
a load measuring unit comprising a load cell for measuring a force applied to a sample stage when the sample is pressed by the crushing member;
a vibration sensor that detects vibration generated from the sample receiving the force; and
a soundproof box housing the drive unit, the crushing member, the load measuring unit and the vibration sensor.

12. The food texture evaluation system according to claim 11, further comprising:
an analytical processing unit configured to analyze data of the load measured by the load measuring unit and data of the vibration detected by the vibration sensor.

13. The food texture evaluation system according to claim 12, wherein the analytical processing unit is configured to display data in such a manner that the data of the load and the data of the vibration are aligned to a common time axis or superimposed.

14. The food texture evaluation system according to claim 11, further comprising:
an image capturing unit configured to capture an image of the sample.
